(19) United States
(12) Patent Application Publication
(10) Pub. No.: US 2026/0057990 A1
(43) Pub. Date: Feb. 26, 2026

(54) SYSTEM INCENTIVIZING AND ADJUSTING DRUG DELIVERY

(71) Applicant: SONARA HEALTH, INC., Dallas, TX (US)

(72) Inventors: Michael GILES, Dallas, TX (US); John Thomas MENCHACA, San Antonio, TX (US); Avinash Prasanna JAYARAMAN, Chicago, IL (US); Dimitri MACRIS, Dallas, TX (US)

(21) Appl. No.: 18/998,867

(22) PCT Filed: Jul. 21, 2023
(86) PCT No.: PCT/US2023/070674

§ 371 Date: Jan. 27, 2025

Related U.S. Application Data
(60) Provisional application No. 63/369,320, filed on Jul. 25, 2022.

(51) Int. Cl. G16H 20/13; G16H 10/60; G16H 40/20; G16H 50/20; G16H 50/30

(52) U.S. Cl. CPC G16H 20/13; G16H 10/60; G16H 40/20; G16H 50/20; G16H 50/30

(57) ABSTRACT

A system for tracking medication compliance having a container having medication and a code on an interior of the container, the code only accessible once the container is opened, a database associating the code with the medication and a patient, a computer in data communication with the database, the computer receiving the code from a patient device, software executing on the computer confirming that the patient took the medication by comparing the code received from the patient device with codes in the database, a healthcare device in data communication with said computer, software executing on the healthcare device receiving confirmation that the patient took the medication from the computer, and software executing on the healthcare device determining a potential adjustment for a future dose of medication for the patient based on the confirmation.

### TECHNICAL FIELD

The disclosure herein generally relates to systems and methods for incentivizing and adjusting drug delivery. More specifically, the disclosure relates to providing incentives for users to take their medication, receive updated/modified prescriptions, participate in clinical trials, and overall lead a healthy lifestyle.

### BACKGROUND

Known systems exist for monitoring drug delivery. For example, U.S. patent application Ser. No. 17/540,717, incorporated herein by reference in its entirety, discloses a system that promotes the tracking of when a patient takes a medication, with an example given for the treatment of Opioid Use Disorder.

However, known systems do not provide for the tracking of a patient's symptoms while taking their medications, and altering their prescriptions and/or treatment plans.

A system providing these functionalities would be useful in at least two scenarios: clinical studies and health insurance. For clinical studies, the ability to track and record additional data regarding a patient can provide valuable clinical insight. For health insurance, knowing how a patient is responding to medication and other treatment may help identify risk and keep a patient from requiring unnecessary procedures. In each of these contexts, rewards may be provided to users for sticking to their treatment plans.

In addition, the present systems facilitate a patient taking their medication. When a prescription is filled, a code may be associated with a medication and stored in a database. Scanning the code allows a patient not only to prove their compliance with their medication, but can allow the system to automatically process refills and adjusted prescriptions by tracking the codes used.

### SUMMARY

A goal of the present teachings is to monitor patient adherence to prescription and non-prescription drug use.

Another goal of the present teachings is to adjust or prescribe prescription and non-prescription drugs to patients.

Another goal of the present teachings to use a remote system for monitoring, adjusting, and prescribing medication.

Another goal of the present teachings to promote clinical trials.

Another goal of the present teachings to provide a system automatically dispatching prescriptions.

In one aspect, a system for tracking medication compliance is provided having a container having medication and a code on an interior of the container. The code is only accessible once the container is opened. A database is provided associating the code with the medication and a patient. A computer is provided in data communication with the database. The computer receives the code from a patient device. Software executing on the computer confirms that the patient took the medication by comparing the code received from the patient device with codes in the database. A healthcare device is in data communication with said computer. Software executing on the healthcare device receives confirmation that the patient took the medication from the computer. Software executing on the healthcare device determines a potential adjustment for a future dose of medication for the patient based on the confirmation.

### BRIEF DESCRIPTION OF THE DRAWINGS

FIG. 1 is a schematic diagram depicting one embodiment of a system for monitoring drug delivery.

FIG. 2 is a flowchart depicting one embodiment of a system for monitoring drug delivery.

### DETAILED DESCRIPTION

The present teachings are described more fully hereinafter with reference to the accompanying drawings, in which the present embodiments are shown. The following description illustrates the present teachings by way of example, not by way of limitation of the principles of the present teachings.

The present teachings have been described in language more or less specific as to structural features. It is to be understood, however, that the present teachings are not limited to the specific features shown and described, since the product herein disclosed comprises preferred forms of putting the present teachings into effect.

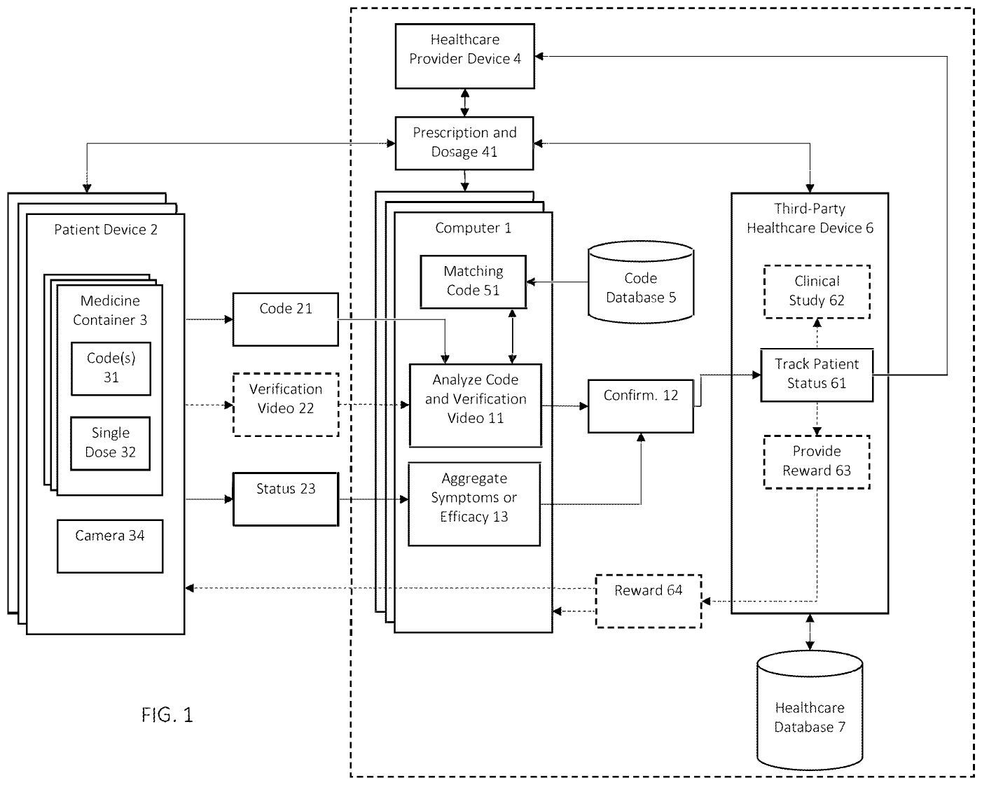

Referring to FIG. 1, shown is one embodiment of a system for monitoring and adjusting drug delivery.

The system may include a computer 1. The computer 1 may be a processor, remote computer, computer server, network, a web server/app or any other computing resource.

The computer 1 may be in data communication with a patient device 2. The patient device 2 may be a computer, laptop, smartphone, tablet or other electronic device capable of transmitting and receiving data.

The patient device 2 may be in data communication with a camera 34 34. The camera 34 may be a part of or separate from a patient device 2. The camera 34 may be a webcam, a phone camera, a laptop camera, or other camera, capable of recording videos. The camera 34 of the patient device 2 may be used to provide pictures, videos, or other information (e.g., the scan of a code) to the computer 1.

The system may also include a medicine container 3. The medicine container 3 may include a medicine 32. For example, the medicine container 3 may include a single dose of a medicine 32. Alternatively, the medicine container 3 may include multiple doses 32.

Medicine 32 may be inside the medicine container 3, although not limited thereto. The medicine 32 may be in any container such as a bottle, pill pouch, package, syringe, canister, or other container for housing medicine. A code 41 may be affixed to the medicine 32 or the container 4. The medicine container 3 and the medicine 32 may be patient-specific and/or dose-specific.

The medicine container 3 may have a code 31. The code 31 may be on the outside of the medicine container 3. The code 31 may be plainly visible on the outside of the medicine container 3, or may be obscured. The code 31 may also be inside the medicine container 3. The code 31 may not be visible, readable, or otherwise knowable until the medicine container 3 is opened. Code 31 may be reprogrammable if medicine container 3 is reusable.

In some embodiments, the medicine container 3 may have two or more codes 31. For example, a first code 31 may be on the outside of the medicine container 3, and the second code 31 may be on an interior of the medicine container 3. It may be required that the first code 31 be irrevocably broken before the second code 31 is accessible. The system may include as many codes 31 as necessary.

The code(s) 31 may be any, one-, two-, or three-dimensional code capable of storing data although not limited thereto. For example, a one-dimensional code may be a barcode, a two-dimensional may be a QR code, and a three-dimensional code may be a 3D array. The medicine container 3 may be made tamper aware through the code 31 which may be a single use barcoded security label.

Embedded in the code 31 may be information about the patient, the medication 32, the dosage, the prescribing doctor, or a time the medication is to be taken. The code 31 may be transmitted to the computer 1 as a code 21. The code 21 may be transmitted in known ways, such as via SMS, MMS, or other data protocols. The code 31 may be encrypted.

The computer 1 may be in data communication with a healthcare provider device 4. The healthcare provider device 4 may be a computer, laptop, smartphone, tablet or other electronic device capable of transmitting and receiving data. The healthcare provider device 4 may be associated with a doctor or other provider who prescribes medicine or treatment plans. Prescriptions and dosage information 41 may be provided to the computer 1, patient device 2, or third-party healthcare devices (discussed below). Healthcare provider device 4 may be used to display information required for a doctor to make the prescription.

The computer 1 may be in data communication with a code database 5. The code database 5 may be a storage drive or array accessible to computer 1, cloud storage, a web-based storage system, or other means for storing data. The code database 5 may send data 51 to the computer 1 or the computer 1 may retrieve data 51 from the code database 5. The code database 5 may store information regarding the system. The code database 5 may include one or more databases.

The code database 5 may include codes 51, which may be associated with medicine 32 which is provided to a patient. Codes 51 may be used by the computer 1 to determine if the code 31 is valid. Code 51 may be used to determine if a code 31 is associated with a specific patient. Code 51 may not exactly match code 31, but may be fed through an equation or algorithm to determine that code 31 is a match. Codes 51 may be associated with medicine 32 that does not require a prescription, such as vitamins or supplements.

The computer 1 may regularly update code database 5 to include new codes 51 from newly-filled prescriptions. The computer 1 may receive new codes from a pharmacy or other medicine-providing organization.

The computer 1 may be in data communication with a third-party healthcare device 6. The third-party healthcare device 6 may be a computer, laptop, smartphone, tablet or other electronic device capable of transmitting and receiving data.

The third-party healthcare device 6 may be associated with a doctor or organization with a healthcare role, such as a medical clinic, hospital, university, or medical insurance company.

The third-party healthcare device 6 may be in data communication with a healthcare database 7. The healthcare database 7 may be a storage drive or array accessible to third-party healthcare device 6, cloud storage, a web-based storage system, or other means for storing data. The healthcare database 7 may send or receive data to or from the third-party healthcare device 6. The healthcare database 7 may store information regarding the system. The healthcare database 7 may include one or more databases.

In various implementations, computer 1, healthcare provider device 4, third-party healthcare device 6, and healthcare database 7 may include some or all of the same computing resources, as shown by the dashed line in FIG. 1.

In use, the system may receive a prescription and a dosage 41 and provide a medicine 32 to a patient in a medicine container 3. When it comes time to take the medicine, the camera 34 may record pictures and/or a video including the code 31, and provide the same to the computer 1 as code 21. Alternatively, the code 21 may be provided as a text string (potentially encoded) or using other known methods.

The camera 34 may also be used to provide a verification video 22 the computer 1. The verification video 22 may also be used to provide the code 21. The verification video 22 may include a picture or video of the patient, who may be promoted to say certain phrases or take certain actions. For example, the patient may be recorded showing the code 32 to the camera 34 and taking the medication 32.

The patient device 23 may also be used to provide a status 23 regarding the patient's condition and medication. For example, the patient may be prompted to provide information regarding diet, exercise, heart rate, sleep, symptoms, or other data that may be clinically valuable. In some embodiments, the patient device 2 may have or be in communication with a device, such as a smart watch, that can provide status information 23. Notably, status information 23 need not be provided at the same time as a code or a verification video; e.g., it may be provided as it is collected. However, a prompt to provide free form text, such as to identify symptoms, may be displayed when a medication is taken.

The computer 1 may execute software 11 to analyze the code 21 and verification video 22. The software 11 may include computer vision and/or other forms of artificial intelligence. For example, the software may include processes for scene reconstruction, object detection, event detection, video tracking, object recognition, 3D pose estimation, learning, indexing, motion estimation, visual servoing, 3D scene modeling, and image restoration.

The software 11 may be used to detect the code 21 in the verification video 22, if it is not provided separately. The computer 1 may query code database 5 and receive matching code 51. Matching code 51 may be used to confirm that the patient has taken medication 32 associated with the proper code 32. For example, codes 21 may be associated with specific times that patients should take medications.

The software 11 may determine a confidence level that the patient successfully took the medication 32. For example, the software 11 may determine that the code 21 is not proper, or that the video does not show the patient ingesting the medication. In such cases, the confidence level may be decreased. If a confidence level is below a certain threshold, the computer 1 may prompt the patient device 2 to provide an explanation for the perceived lack of compliance. Such explanation may be provided as status information 23.

The computer 1 may also have software 13 aggregating status information 23 provided by the patient device 2. Such status information 23 may be stored in a database associated with the patient until required.

The computer 1 may also track when a patient is expected to take a dose of medicine, and provide a confirmation 12 that the patient did not take a dose or upload a code 21 or video 22.

The computer 1 may provide a confirmation 12 that the patient took their medication 32 to the third-party healthcare device 6. The confirmation 12 may include the code 21, verification video 22, and aggregated status information 13.

The third-party healthcare device 6 may use the confirmation 12 to track patient status 61. Tracking patient status 61 may include having a doctor or other practitioner review the confirmation 12, for example, if it was determined that the confidence level was below a certain threshold, or at random.

Tracking patient status 61 may include software for anonymizing and storing patient data in a healthcare database 7. As confirmations 12 are received over time, they may be compared to anonymized data and used to provide suggestions to healthcare providers (through healthcare provider device 4) regarding suggested medications, dosages, and treatments. The software may include artificial intelligence software, such as machine learning, pattern recognition, regression, sequence labelling, and/or parsing. The software may also be computer-aided detection or computer-aided diagnosis software.

The patient status may be provided to the healthcare provider device 4. The healthcare provider may chose to alter the prescription or dosage based on the patient status 61. A new prescription and dosage 41 may be provided to the computer 1, which may interface with a pharmacy or other medicine-supplying organization to associate codes 31 on medicine containers for patients. The computer 1 may cause the new medicine to be shipped to the patient in some instances, or provide a notification to the patient device 2 that the medicine is available for pickup.

The third-party healthcare device 6 may aggregate the confirmations to use them as part of a clinical study 62. Such information for clinical studies 62 may be anonymized.

The third-party healthcare device 6 may also have software 63 for determining rewards 64 to provide to patients for the adherence to their treatment plan. Rewards 64 may be provided directly to the patient device 2 or to the patient through computer 1.

Data in the system may be encrypted or anonymized as known in the art.

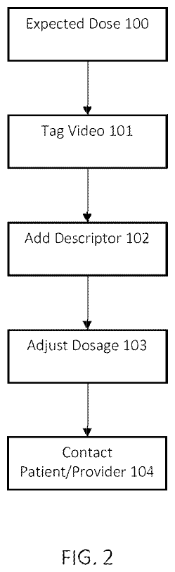

A workflow associated with an embodiment the system is shown in FIG. 2. When an expected dose 100 is to occur, it may first be determined whether the corresponding data (such as code 31 and verification video 32) have been uploaded. If not, the dose may be classified as not having been taken, and contact may be attempted with the patient.

If a video is uploaded, it may be tagged 101 as “unreviewed,” “reviewed,” “for follow up” or “last dose. ” Unreviewed videos may be put into a queue for review. Reviewed videos showing successful dosing may be rewarded with a congratulatory contact with the patient, sharing video of the dosing with a physician, or saving the recording for later recall by the physician. Videos marked as “follow-up” may include videos lacking a view of the dosing event, a technical bug, or an otherwise “unacceptable dosing event,” such as if someone else took the medication. If one of these events occurs, the reviewer of an email may provide a description 102 of the event, and flag it for contact with the patient, doctor, or clinic. A final dosage event of a regimen will trigger a prompt for the doctor or clinic to provide a new regimen.

The system or the doctor may determine to adjust dosage or the medication regimen 103. Finally, the patient and/or healthcare provider may be contacted 104 with information regarding the new medication regimen.

Although the invention has been illustrated and described herein with reference to a preferred embodiment and a specific example thereof, it will be readily apparent to those of ordinary skill in the art that other embodiments and examples may perform similar functions and/or achieve user experiences. All such equivalent embodiments and examples are within the spirit and scope of the present invention, are contemplated thereby, and are intended to be covered by the disclosure.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

What is claimed is:

1. A system for tracking medication compliance, comprising:
a container having medication and a code on an interior of the container, the code only accessible once the container is opened;
a database associating the code with the medication and a patient;
a computer in data communication with the database;
the computer receiving the code from a patient device;
software executing on the computer confirming that the patient took the medication by comparing the code received from the patient device with codes in the database;
a healthcare device in data communication with the computer;
software executing on the healthcare device receiving confirmation that the patient took the medication from the computer; and
software executing on the healthcare device determining a potential adjustment for a future dose of medication for the patient based on the confirmation.

2. The system of claim 1, wherein the code is transmitted by the patient device to the computer via SMS.

3. The system of claim 1, wherein the container comprises a packet and the code is printed on an inside of a packet.

4. The system of claim 1, wherein the code is associated with a time for the patient to take the medication.

5. The system of claim 4, the software executing on the computer only confirming that the patient took the medication if the computer receives the code near the time for the patient to take the medication.

6. The system of claim 1, further comprising:
the healthcare device receiving a medical status;
software executing on the healthcare device determining a potential adjustment for the future dose of medication for the patient based on the medical status.

7. The system of claim 6, wherein the medical status of the patient comprises a self-report about pain, fatigue, discomfort or symptoms.

8. The system of claim 6, wherein the medical status of the patient is test results, bloodwork results, or healthcare provider notes.

9. The system of claim 6, wherein the medical status is generated by a wearable device in data communication with the patient device.

10. The system of claim 6, further comprising software executing on the healthcare device for anonymizing the medical status for storage in a second database for use in making future adjustments to prescriptions.

11. The system of claim 1, wherein the software for determining a potential adjustment uses machine learning.

12. The system of claim 1, the healthcare device generating a reward based on the confirmation;
the reward received by the patient device.

13. The system of claim 1, the healthcare device transmitting the potential adjustment to a device associated with the patient's prescribing doctor;
wherein the device associated with the patient's prescribing doctor generates a new prescription for the patient.

14. The system of claim 13, wherein the new prescription is sent to the computer for fulfillment.

15. The system of claim 1, wherein the healthcare device is associated with a clinical study.

16. The system of claim 1, wherein the healthcare device is associated with a health insurance company.

17. The system of claim 1, the patient device having a camera for scanning the code.

18. The system of claim 1, the patient device having a camera for taking video of the patient administering the medication;
the computer receiving the video of the patient taking the medication;
software executing on the computer using machine vision to confirm whether the patient took the medication.

19. The system of claim 1, the container having a second code on an outside of the container, wherein the patient is required to break the second code to access the first code;
the database associating the second code with the medication and the patient;
the computer receiving the second code from the patient device;
software executing on the computer confirming that the patient took the medication by comparing the second code received from the patient with codes in the database.

20. The system of claim 1, wherein the code is encrypted.
